(19) United States
(12) Patent Application Publication
(10) Pub. No.: US 2010/0170510 A1
(43) Pub. Date: Jul. 8, 2010

(54) WATER RESERVOIR BAFFLE

(75) Inventor: Alexander Pieri, Box Hill North (AU)

Correspondence Address: SUGHRUE MION, PLLC, 2100 PENNSYLVANIA AVENUE, N.W., SUITE 800, WASHINGTON, DC, 20037, US

(73) Assignee: COMPUMEDICS MEDICAL INNOVATION PTY LTD, Abbotsford, Victoria (AU)

(21) Appl. No.: 12/663,192

(22) PCT Filed: Jun. 2, 2008
(86) PCT No.: PCT/AU2008/000766

§ 371 Date: Dec. 4, 2009

(30) Foreign Application Priority Data
Jun. 4, 2007 (AU) 2007902959

(51) Int. Cl. A61M 16/16

(52) U.S. Cl. 128/203.17

(57) ABSTRACT

TThe present invention provides a water reservoir for humidification of the gas delivered by a gas-delivery device. The water reservoir comprises of a container having at least one opening for gas movement, and a means to prevent water escape through the aperture while allowing passage of gas to be humidified and delivered to a subject. Preferably the means to prevent water escape is a baffle. Preferably the baffle is disposed opposite at least a portion of the opening. The baffle may be continuous with a wall of the container. The container may be comprised of a deformable material.

### FIELD OF THE INVENTION

This invention relates to water reservoirs for humidification of air streams in gas-delivery devices.

### BACKGROUND

There are a number of devices available for the delivery of breathable gas, to a patient for the treatment of sleep disordered breathing. For example, in the treatment of obstructive sleep apnoea, air is often delivered at continuous positive air pressure (CPAP) wherein air is supplied continuously at a pressure greater than ambient to the nose of a sleeping patient through a mask to keep the patient's airways open for effective respiration.

It is important for continuous therapeutic benefit from pressurised air that the air and other gases to be delivered at a pressure and flow rate appropriate for the desired breathing pattern. Further patient comfort is enhanced if the air is humidified. Patent application published as WO 2007/045017 directed toward an apparatus for delivery of pressurised gas describes an advanced example of a CPAP device and is incorporated herein by reference. The CPAP device incorporates a water reservoir for humidifying air, the reservoir storing water that can be evaporated by heating by the conduction of heat from a heater to the water. The CPAP device described in WO 2007/045017 conveniently operates horizontally or vertically. However, the flexibility in orientation of the invention restricts the volume of water the reservoir can store relative to the total volume available in the reservoir because of the possibility of spillage. In turn, this limitation limits the total volume of air that can be humidified during a treatment period without needing to refill the reservoir. Treatment periods optimally extend for the duration of a sleep period of a user without the reservoir running dry.

What is needed is a water reservoir for a CPAP device, or other gas delivery device that incorporates a heater for humidification of delivered air, that optimises the available water-storing capacity of the reservoir.

### BRIEF DESCRIPTION OF THE FIGURES

FIG. 1a shows a transverse section of a gas-delivery device incorporating a water reservoir positioned for operation.

FIG. 1b shows a top view of a gas-delivery device incorporating a water reservoir positioned for operation.

FIG. 2 shows an example of a baffle that for incorporation in a water reservoir for preventing leakage.

FIG. 3 shows a transverse section of a water vessel incorporating a baffle for preventing leakage.

FIG. 4 shows a perspective view of a baffle disposed on the inside surface of a water reservoir.

FIG. 5 shows a transverse section of a water reservoir incorporating a baffle.

FIG. 6 shows a transverse section of a water reservoir incorporating a baffle disposed to be filled with water.

FIG. 7 shows a transverse section of a water reservoir incorporating a baffle disposed for insertion into a gas-delivery device.

### SUMMARY OF THE INVENTION

Water reservoirs for therapeutic gas-delivery CPAP devices ideally hold enough water to humidify the air delivered to a patient for long periods of time, such as a full night's sleep. A water reservoir should be easily removable from a gas delivery device for cleaning, replacing, or filling. The volume of a water reservoir is constrained by the volume of space available within the enclosed spatial boundaries of the gas-delivery device. Ideally, a water reservoir comprises of material that is relatively cheap and easy to manufacture into the shape and volume available in the gas-delivery device. It is an object of the invention to provide for water reservoir for a gas-delivery device with that has increased volume for water storage. It is an object of the invention to provide water reservoir for a gas-delivery device that has reduced risk of water spillage during operation.

In one aspect the invention provides a water reservoir for a gas-delivery device comprising a container having an opening and a means for reducing water escape through said opening, said means being disposed to allow passage of gas. Preferably, the means for reducing water escape is disposed opposite at least a portion of the opening. Preferably the means for reducing water escape is a baffle. Preferably, the means for reducing water escape is continuous with the wall of the container. Preferably the reservoir comprises of deformable material. Preferably, the baffle comprises of an extension of a wall of the container.

### DETAILED DESCRIPTION OF THE INVENTION AND BEST METHOD OF PERFORMANCE

In this document a reference to “comprising” is a reference to “including”, where both words are used in a context that is not limiting. The invention is most easily understood with reference to the accompanying figures. It will be understood that the figures are intended to be illustrative embodiments of the invention and that the scope of the invention as defined in the claims includes further embodiments not so illustrated.

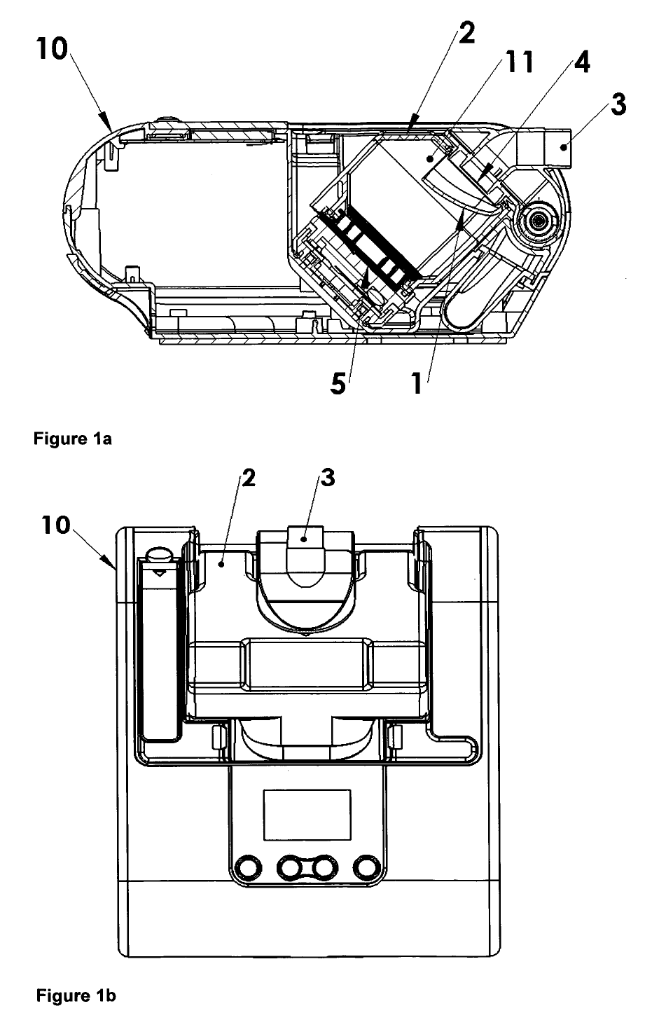

The present invention provides a means for preventing water escape through spilling or leaking from the opening of water reservoir through which gases flow to be humidified before delivery to a subject. Preferably, the means is a baffle. FIGS. 1a and 1b show two perspective views of a gas-delivery device 10 incorporating a water reservoir 2 having a baffle 1. A transverse section in side perspective of the gas-delivery device 10 is shown in FIG. 1a. It can be seen that the water reservoir in this embodiment is tilted. A conduit 3 engages with the water reservoir 2 at an aperture 4 in the water reservoir 2. A top view of the gas-delivery device 10 is shown in FIG. 1b. The volume enclosed by the water reservoir includes a headspace 11 of gas or air. Air or gas is pumped into the headspace by the pump (not shown) of the gas-delivery device 10. The water is heated by conduction of heat produced by a heat source 5, which may be a heating plate, coil or some other suitable heating means. The heat source 5 may extend through the wall 6 of the water reservoir and into the water. The temperature of the water determines the water vapour pressure and humidity of the air or gas in the head space 11. The water reservoir preferably includes a single aperture 4 for ingress and egress of air. The scope of the invention includes that the air may and enter exit the headspace through different apertures. Each aperture may be opposed by a means 1 for preventing water leaking or spilling through the aperture and also allows the water level to be higher than might otherwise occur in the absence of the means 1. The prevention of leaking or spilling is most effective when each aperture is opposed with a baffle in the correct orientation. The maximum level of water allowable in the water reservoir will be determined by the height of the edge of the highest baffle in each orientation of the gas-delivery device.

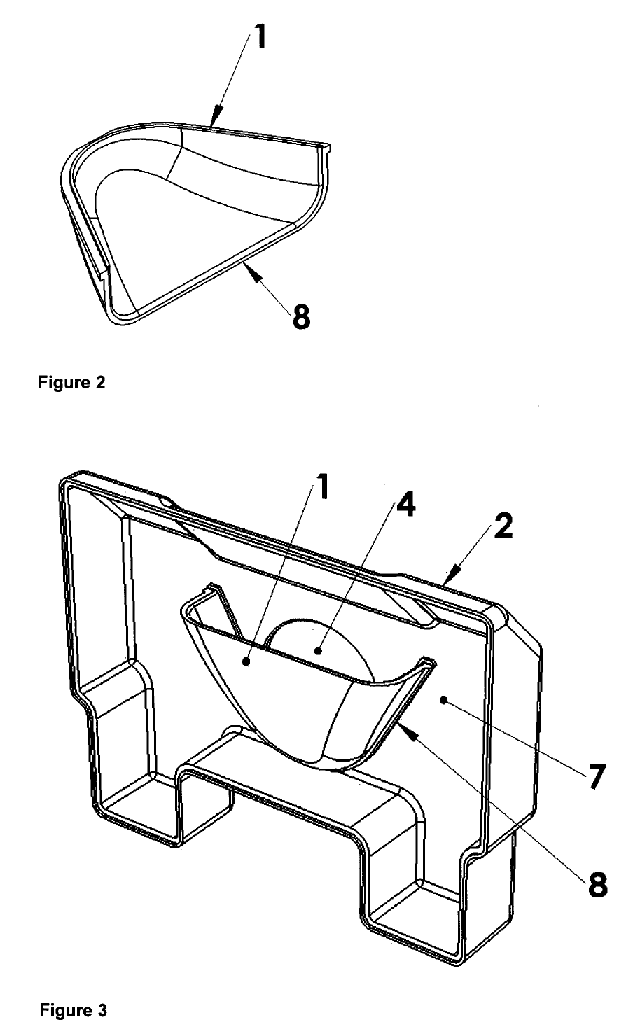

An embodiment of a leakage prevention means is shown in FIG. 2 as a baffle. In this embodiment, the baffle 1 is disposed in fixed position to the adjacent wall 7 of the water reservoir to the aperture. Preferably the baffle is bonded to the wall along an engaging portion 8 of the baffle. FIG. 3 shows an embodiment of the baffle 1 shown in FIG. 2, the baffle being bonded in position to a wall of the water reservoir. It is important that the engaging portion of baffle is engaged in a leak-proof manner along at least the portion of the length of the baffle according to the edge of the aperture.

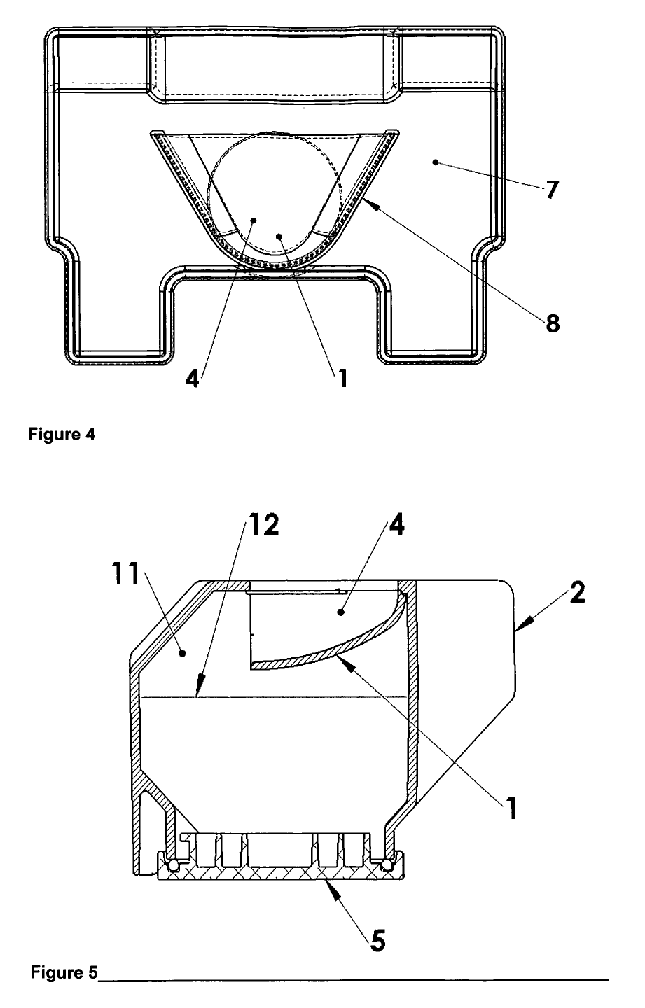

Preferably the engaging portion 8 of the baffle follows the lower edge of the aperture 4 before fanning out at a symmetrical angle and finishing level with the top of the container opening as shown in FIG. 4.

The surface profile of a baffle is important to ensure minimal resistance to airflow through the aperture 4 and into the headspace 6 of the water reservoir. The preferred embodiment of the surface profile of a baffle is shown in FIG. 5, which shows a transverse section a baffle 1 forming a gently curved for the purpose of guiding the air stream into and out of the water reservoir with a minimum of resistance. Most preferably the distance between the baffle 1 and the wall 7 of the reservoir is about 18 mm. Other shapes for the baffle are possible, but it is possible the shapes will be more restrictive of air flow notwithstanding an increased volume of water being held in the reservoir with other baffle shapes. Preferably, the edge of the baffle which is opposite the engaging portion is about level with the furthest edge of the aperture 4, however, it is possible that the edge may extend more or less than the preferred embodiment.

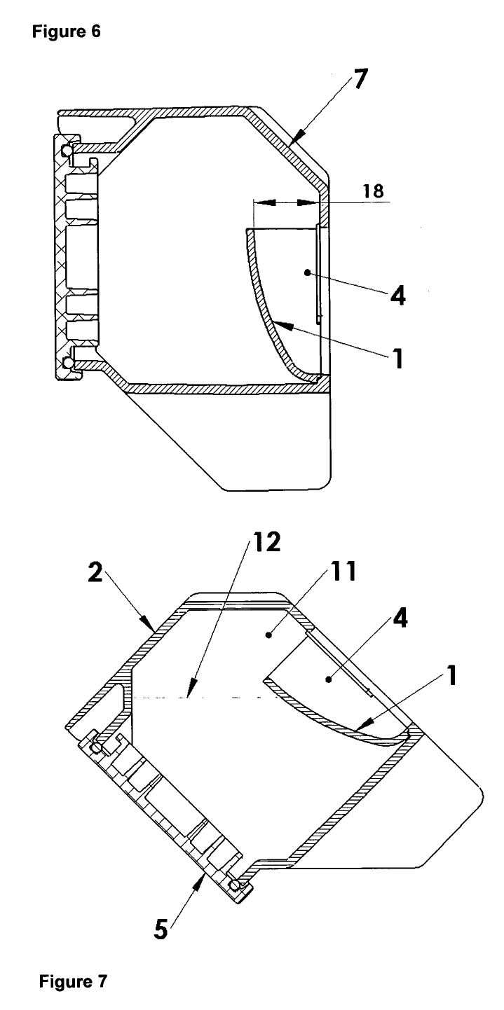

An advantage of the invention can be seen with reference to FIGS. 6 and 7, which indicate sequentially the ease with which the water reservoir 2 can be filled with water and then positioned for operation in a gas-delivery device with minimal spillage of water. In FIG. 6 the water reservoir is shown ready to receive a recharge of water, being disposed for that the aperture 4 is parallel with the surface on which the reservoir sits. In FIG. 7, the water reservoir is shown at a slightly tilted position in the gas-delivery device. In each of FIGS. 6 and 7 the water level, shown as a dashed line 12. It can be seen easily in FIG. 7 that baffle 1 prevents the spillage of water through the aperture 4 in multiple orientations of the water reservoir.

1. A water reservoir for a gas-delivery device comprising a container having an opening and a means for reducing water escape through said opening, said means being disposed to allow passage of a gas.

2. The water reservoir according to claim 1 wherein the means for reducing the water escape is disposed opposite at least a portion of the opening.

3. The water reservoir according to claim 1, wherein the means for reducing water escape is a baffle.

4. The water reservoir according to claim 1, wherein the means for reducing water escape is continuous with the wall of the container.

5. The water reservoir of claim 1 wherein the reservoir comprises of deformable material.

6. The water reservoir of claim 5, wherein the baffle comprises of an extension of a wall of the container.

7. The water reservoir of any of claim 2, wherein the means for reducing water escape is a baffle.

8. The water reservoir according to any of claim 7, wherein the means for reducing water escape is continuous with the wall of the container.

9. The water reservoir according to any of claim 2, wherein the means for reducing water escape is continuous with the wall of the container.

10. The water reservoir according to any of claim 3, wherein the means for reducing water escape is continuous with the wall of the container.
